(19) United States
(12) Patent Application Publication
(10) Pub. No.: US 2026/0108559 A1
(43) Pub. Date: Apr. 23, 2026

(54) COMPOSITIONS OF EXTRACELLULAR VESICLES AND METHOD USING THE SAME

(71) Applicant: Ascension Medical Biotechnology Co., Ltd., Taipei City (TW)

(72) Inventors: Ju-Sheng Shieh, Taipei City (TW); Yu-Tang Chin, Taipei City (TW)

(73) Assignee: Ascension Medical Biotechnology Co., Ltd., Taipei City (TW)

(21) Appl. No.: 19/366,546

(22) Filed: Oct. 23, 2025

Related U.S. Application Data
(60) Provisional application No. 63/710,610, filed on Oct. 23, 2024.

(30) Foreign Application Priority Data
Oct. 22, 2025 (TW) 114140979

(51) Int. Cl. A61K 35/28; A61K 31/7105; A61L 27/36; A61L 27/38; B82Y 5/00; C12N 5/0775

(52) U.S. Cl. CPC A61K 35/28; A61K 31/7105; A61L 27/365; A61L 27/3834; C12N 5/0668; A61L 2430/02; B82Y 5/00; C12N 2500/30; C12N 2501/155

(57) ABSTRACT

The present disclosure provides a composition comprising primed extracellular vesicles and a biologically or pharmaceutically acceptable carrier, wherein the primed extracellular vesicles comprise an upregulated miRNA, and a fold change of the upregulated miRNA of the primed extracellular vesicles compared to miRNA of naturally occurring extracellular vesicles is greater than 1. The present disclosure further provides a method for preventing, treating, or ameliorating a bone defect or a dental defect comprising administering the composition of the present disclosure to a subject in need thereof. The present disclosure also provides a method for producing the composition. The method comprises cultivating a stem cell in a culture medium containing a Polygonum multiflorum Thunb extract and at least one selected from the group consisting of baicalin and bone morphogenetic protein 6 to obtain a cell culture.

### CROSS REFERENCE TO RELATED APPLICATIONS

This application claims the benefit of U.S. Provisional Application No. 63/710,610, filed on Oct. 23, 2024. The content of the application is incorporated herein by reference.

### BACKGROUND OF THE INVENTION

### 1. Field of the Invention

The present disclosure relates to a composition comprising extracellular vesicles, and more particularly to extracellular vesicles produced by the special process and the use of the extracellular vesicles.

### 2. Description of the Prior Art

Stem cell-based therapy is a treatment approach that uses stem cells per se to repair, regenerate, rejuvenate, or replace damaged or dysfunctional tissues and organs in a subject in need thereof. Stem cell-based therapy holds potential for regenerative medicine and personalized medicine since stems cells are able to self-renew and develop into various cell types. However, stem cell-based therapy still faces several challenges, including transplant rejection, tumor transformation, limited sources, and ethical issues, which limit its clinical application.

Compared to cells per se, extracellular vesicles (EVs) from the cells offer advantages such as low immunogenicity, non-tumorigenicity, high clinical safety, and low ethical risk. The International Society of Extracellular Vesicles (ISEV) defines EVs as “a collective term for particles secreted by cells that are composed of a lipid bilayer and do not possess the ability to replicate independently, meaning they lack a functional nucleus.” Generally, EVs can be classified into three main categories based on their biogenesis mechanisms and particle sizes: apoptotic bodies (approximately 50-2000 nm in diameter), microvesicles (approximately 150-1000 nm in diameter), and exosomes (approximately 30-150 nm in diameter). Due to the size differences among extracellular vesicles, nomenclature often categorizes them as small EVs (sEVs, <200 nm) or medium/large EVs (>200 nm). Since EVs transport signaling molecules such as mRNA, microRNA (miRNA), circRNA, lncRNA, proteins, DNA fragments, and lipids, facilitating signal transmission between cells through internalization, ligand-receptor interactions, or membrane fusion, they play critical roles in physiological and pathological processes.

Compared to the effects of naïve MSC-derived EVs (i.e., naturally occurring extracellular vesicles by the mesenchymal stem cells), which can vary due to the culture conditions of MSCs, numerous studies have shown that EVs from primed or induced MSCs contain different levels of metabolites that may be involved in therapeutic modulation. However, there is still an unmet need in the art to develop an approach to produce primed EVs or guided EVs from MSCs to provide new pathways and solutions for the treatment of various diseases by enhancing the performance of EVs bioactive molecules and improving the therapeutic effect.

### SUMMARY OF THE INVENTION

In one aspect, the present disclosure provides a composition comprising primed extracellular vesicles and a biologically or pharmaceutically acceptable carrier, wherein the primed extracellular vesicles comprise an upregulated miRNA, and a fold change of the upregulated miRNA of the primed extracellular vesicles compared to miRNA of naturally occurring extracellular vesicles is greater than 1.

In some embodiments, the primed extracellular vesicles of the present disclosure are cultivated from mesenchymal stem cells.

In some embodiments, the mesenchymal stem cells of the present disclosure are selected from the group consisting of avian mesenchymal stem cells (hereinafter referred as, AMSCs), human umbilical cord mesenchymal stem cells (hereinafter referred as, hUC-MSCs), human placental mesenchymal stem cells (hereinafter referred as, hPLAMSCs), adipose-derived mesenchymal stem cells, bone-marrow mesenchymal stem cells, dental pulp mesenchymal stem cells, gingival mesenchymal stem cells, and a combination thereof.

In some embodiments, the fold change of the upregulated miRNA of the primed extracellular vesicles of the present disclosure compared to the miRNA of the naturally occurring extracellular vesicles is greater than 1, and the upregulated miRNA of the primed extracellular vesicles is selected from the group consisting of miR-20a-5p, miR-26a-5p, miR-26b-5p, miR-29a-3p, miR-92a-3p, miR-200b-3p, miR-200c-3p, miR-204-5p, miR-210-3p, miR-214-3p, miR-218-5p, miR-222-3p, miR-335-5p, and a combination thereof.

In some embodiments, the fold change of the upregulated miRNA of the primed extracellular vesicles of the present disclosure compared to the miRNA of the naturally occurring extracellular vesicles is greater than 2, and the upregulated miRNA of the primed extracellular vesicles is selected from the group consisting of miR-29a-3p, miR-92a-3p, miR-200b-3p, miR-200c-3p, miR-204-5p, miR-214-3p, miR-335-5p, and a combination thereof.

In some embodiments, the fold change of the upregulated miRNA of the primed extracellular vesicles of the present disclosure compared to the miRNA of the naturally occurring extracellular vesicles is greater than 5, and the upregulated miRNA of the primed extracellular vesicles is selected from the group consisting of miR-200b-3p, miR-204-5p, miR-335-5p, and a combination thereof.

In some embodiments, the fold change of the upregulated miRNA of the primed extracellular vesicles of the present disclosure compared to the miRNA of the naturally occurring extracellular vesicles is greater than 13, and the upregulated miRNA of the primed extracellular vesicles is miR-335-5p.

In some embodiments, the fold change of a downregulated miRNA of the primed extracellular vesicles compared to miRNA of naturally occurring extracellular vesicles is less than 1.

In some embodiments, the downregulated miRNA of the primed extracellular vesicles is selected from the group consisting of miR-30b-5p, miR-30e-5p, miR-31-5p, miR-34a-5p, miR-126-3p, miR-138-5p, miR-223-3p, and a combination thereof.

In some embodiments, the extracellular vesicle of the present disclosure is an apoptotic body, a microvesicle, an exosome, or any combination thereof.

In some embodiments, the extracellular vesicles of the present disclosure comprise a polydisperse population of particles with a diameter ranging from about 30 nm to about 200 nm.

In some embodiments, the composition of the present disclosure is formulated into an injectable solution, hydrogel, scaffold composite, or implant coating.

In one aspect, the present disclosure further provides a method for preventing, treating, or ameliorating a bone defect or a dental defect comprising administering the composition of the present disclosure to a subject in need thereof.

In some embodiments, the bone defect is selected from the group consisting of long bone fracture, spinal fusion augmentation, osteoporotic fracture and non-union defect.

In some embodiments, the dental defect is selected from the group consisting of alveolar ridge preservation, periodontal intrabony defect, furcation defect, and peri-implantitis-associated bone loss.

In one embodiment, the administering of the present disclosure comprises topical administration.

In one embodiment, the administering of the present disclosure comprises local administration.

In some embodiments, the composition of the present disclosure exhibits enhanced osteogenic or cementogenic bioactivity.

In yet another aspect, the present disclosure further provides a method for producing the composition of the present disclosure and the method comprises cultivating a stem cell in a culture medium containing a Polygonum multiflorum Thunb extract and at least one selected from the group consisting of baicalin and bone morphogenetic protein 6 to obtain a cell culture; obtaining the extracellular vesicle from the cell culture; and mixing a biologically or pharmaceutically acceptable carrier with the extracellular vesicle.

In some embodiments, the Polygonum multiflorum Thunb extract comprises 2,3,4′,5-tetrahydroxystilbene-2-O-β-D-glucoside (hereinafter referred as THSG).

In some embodiments, the Polygonum multiflorum Thunb extract of the present disclosure has a concentration of about 0.1 μM to about 50 μM.

In some embodiments, the cultivation of the MSCs of the present disclosure is performed for a time period ranging from 24 hours to 96 hours.

The details of one or more embodiments of the invention are set forth in the description below. Other features or advantages of the present disclosure will be apparent from the following drawings and detailed description of several embodiments, and also from the appended claims.

### BRIEF DESCRIPTION OF THE DRAWINGS

The patent or application file contains at least one drawing executed in color. Copies of this patent or patent application publication with color drawings will be provided by the Office upon request and payment of the necessary fee. The following drawings form part of the present specification and are included to further demonstrate certain aspects of the present disclosure. The present disclosure may be better understood by reference to one or more of these drawings in combination with the detailed description of specific embodiments presented herein.

FIG. 1 illustrates the effect of bio-pulsed extracellular vesicles derived from TBA-stimulated MSCs (BPEV-TBA) on the proliferation of primary human osteoblasts (OB-F), compared to naïve MSC-derived EVs and untreated controls. Cells were incubated for 72 hours, and proliferation was quantified using a CCK-8 assay.

FIG. 2 illustrates the effect of bio-pulsed extracellular vesicles derived from TBA-stimulated MSCs (BPEV-TBA) on the proliferation of human alveolar bone cells, compared to naïve MSC-derived EVs and untreated controls. Cells were cultured for 72 hours, and proliferation was quantified using a CCK-8 assay.

FIG. 3 illustrates representative wound-healing scratch assays of human osteoblasts (OB-F) treated with control medium, naïve MSC-derived EVs, or bio-pulsed EVs (BPEV-TBA). Cells were cultured using ibidi Culture-Inserts to create standardized gaps, and images were captured at baseline (0 hr) and after 24 hours of incubation.

FIG. 4 illustrates alkaline phosphatase (ALP) staining of human osteoblasts (OB-F) cultured in osteogenic induction medium supplemented with either control medium, naïve MSC-derived EVs, or bio-pulsed EVs (BPEV-TBA), each applied at a concentration of 1×10⁸ particles/mL. Cells were maintained for 3, 7, and 14 days, and representative images show progressive osteogenic differentiation, with staining intensity varying according to treatment condition.

FIG. 5 illustrates Alizarin Red staining of human osteoblasts (OB-F) cultured in osteogenic induction medium supplemented with control medium, naïve MSC-derived EVs, or bio-pulsed EVs (BPEV-TBA), each applied at a concentration of 1×10⁸ particles/mL. Representative images at 7 and 14 days show calcium nodule formation and mineral deposition under the different treatment conditions.

FIG. 6A, FIG. 6B, FIG. 6C, and FIG. 6D illustrate the relative mRNA expression levels of osteogenic marker genes in human osteoblasts (OB-F) cultured in osteogenic induction medium supplemented with control medium, naïve MSC-derived EVs, or bio-pulsed EVs (BPEV-TBA), each applied at a concentration of 1×10⁸ particles/mL. After 3 days of induction, RNA was extracted and analyzed by quantitative PCR. The bar graphs show expression levels of RUNX2, ALP, osteocalcin (OC), and COL1A1, normalized to 18s rRNA.

FIG. 7 illustrates representative phase-contrast microscopy images of U937 cells induced toward osteoclast differentiation using phorbol 12-myristate 13-acetate (PMA) in combination with vitamin D3, in the presence of control medium, naïve MSC-derived EVs, or bio-pulsed EVs (BPEV-TBA) at doses of 1×10⁴, 1×10⁶, and 1×10⁸ particles/mL. Images were captured at day 7 post-induction. Red arrows indicate multinucleated osteoclast-like cells formed under the respective treatment conditions.

FIG. 8 illustrates representative micro-computed tomography (micro-CT) reconstructions of periodontal defects in rats treated with control medium, naïve MSC-derived EVs, or bio-pulsed EVs (BPEV-TBA) at different doses. Following surgical creation of standardized periodontal defects, defects were filled with Matrigel containing the designated EV preparation. After 4 weeks of healing, maxillary specimens were harvested and analyzed by micro-CT. The reconstructed images show bone regeneration in the defect regions.

FIG. 9 illustrates the quantitative analysis of the bone support ratio (BC/AC×100%) in each treatment group.

FIG. 10A, FIG. 10B, and FIG. 10C illustrate quantitative micro-computed tomography (micro-CT) analysis of periodontal defects in rats treated with control medium, naïve MSC-derived EVs, or bio-pulsed EVs (BPEV-TBA) at different doses. Four weeks after defect creation and local filling with Matrigel containing the designated EVs, harvested maxillary specimens were analyzed. The bar graphs show (left) bone volume fraction (BV/TV, %), (middle) trabecular thickness (Tb.Th, μm), and (right) trabecular separation (Tb.Sp, μm), comparing each treatment group.

FIG. 11 illustrates representative micro-computed tomography (micro-CT) reconstructions of extraction sockets in rats treated with control medium, naïve MSC-derived EVs, or bio-pulsed EVs (BPEV-TBA) at different doses. Following tooth extraction, sockets were filled with Matrigel containing the designated EV preparation. After 4 weeks, specimens were harvested and scanned by micro-CT. The micro-computed tomography shows three-dimensional reconstructions and sectional views of extraction sockets. The lower panels present quantitative measurements of bone volume fraction (BV/TV, %), trabecular thickness (Tb.Th, μm), and trabecular separation (Tb.Sp, μm) among the different treatment groups.

FIG. 12A, FIG. 12B, and FIG. 12C illustrate quantitative measurements of bone volume fraction (BV/TV, %), trabecular thickness (Tb.Th, μm), and trabecular separation (Tb.Sp, μm) among the different treatment groups.

FIG. 13 illustrates the effect of extracellular vesicle treatments on the proliferation of murine cementoblast OCCM30 cells. Cells were incubated with control medium, naïve MSC-derived EVs, or bio-pulsed EVs stimulated with TBMP6 (BPEV-TBMP6) at doses of 1×10⁶ and 1×10⁸ particles/mL for 48 hours. Cell proliferation was assessed using a CCK-8 assay, and results are expressed as relative proliferation percentage compared to control.

FIG. 14A, FIG. 14B, FIG. 14C, and FIG. 14D illustrate the relative mRNA expression levels of osteogenic and cementogenic marker genes in murine cementoblast OCCM30 cells after mineralization induction for 3 days. Cells were treated with control medium, naïve MSC-derived EVs, or bio-pulsed EVs stimulated with TBMP6 (BPEV-TBMP6), each applied at a concentration of 1×10⁸ particles/mL. Quantitative PCR analysis shows the expression of RUNX2, osteocalcin (OC), cementum attachment protein (CAP), and dentin sialophosphoprotein (DSPP), normalized to 18s rRNA.

### DETAILED DESCRIPTION

Those skilled in the art will readily observe that numerous modifications and alterations of the present disclosure may be made while retaining the teachings of the disclosure described herein. Accordingly, the embodiments described are intended to cover the modifications and alterations within the scope of the present disclosure, rather than to limit the present disclosure. The scope of the claims therefore should be accorded the broadest interpretation so as to encompass all such modifications and alterations.

In this disclosure, all terms including descriptive or technical terms which are used herein should be construed as having meanings that are obvious to one of ordinary skill in the art. However, the terms may have different meanings according to an intention of one of ordinary skill in the art, case precedents, or the appearance of new technologies. Also, some terms may be arbitrarily selected by the applicant, and in this case, the meaning of the selected terms will be described in detail in the descriptions of the present disclosure. Thus, the terms used herein are defined based on the meaning of the terms together with the descriptions throughout the specification.

As used herein, the singular forms “a,” “an,” and “the” are intended to include the plural forms, unless the context clearly indicates otherwise. The terms “includes,” “including,” “comprises,” and “comprising” are used in either the detailed descriptions and/or the claims, and such terms are intended to be inclusive in a manner of not excluding others, such as other components, materials, steps, etc. The terms “sec,” “min,” and “hr” as used herein are abbreviations of “second,” “minute,” and “hour.”

As used herein, the term “comprising,” “comprises” “include,” “including,” “have,” “having,” “contain,” “containing,” and any other variations thereof are intended to cover a non-exclusive inclusion. For example, when describing an object “comprises” a limitation, unless otherwise specified, it may additionally include other ingredients, elements, components, structures, regions, parts, devices, systems, steps, or connections, etc., and should not exclude other limitations.

Where ranges are given, endpoints are included. Furthermore, unless otherwise indicated or otherwise evident from the context and understanding of one of ordinary skill in the art, values that are expressed as ranges can assume any specific value or sub-range within the stated ranges in different embodiments of this disclosure, unless the context clearly dictates otherwise.

As used herein, the term “about” generally referring to the numerical value meant to encompass variations of ±20%, ±10%, ±5%, ±1%, ±0.5%, or ±0.1% from a given value or range. Such variations in the numerical value may occur by, e.g., the experimental error, the typical error in measuring or handling procedure for making compounds, compositions, concentrates, or formulations, the differences in the source, manufacture, or purity of starting materials or ingredients used in the present disclosure, or like considerations. Alternatively, the term “about” means within an acceptable standard error of the mean when considered by a person having ordinary skill in the art. Unless otherwise expressly specified, all of the numerical ranges, amounts, values and percentages such as those for quantities of materials, durations of time periods, temperatures, operating conditions, ratios of amounts, and the likes disclosed herein should be understood as modified in all instances by the term “about.”

The numeral ranges used herein are inclusive and combinable, any numeral value that falls within the numeral scope herein could be taken as a maximum or minimum value to derive the sub-ranges therefrom. For example, the numeral range “0.1 μM to 50 μM” includes any sub-ranges between the minimum value of 0.1 μM to the maximum value of 50 μM, such as the sub-ranges from 0.1μ M to 40μ, from 10μ M to 50μ, from 1μ M to 20μ M and so on. In addition, a plurality of numeral values used herein can be optionally selected as maximum and minimum values to derive numerical ranges. For instance, the numerical ranges of 0.5 μM to 5 μM, 0.5 μM to 50 μM, and 5 μM to 50 μM can be derived from the numeral values of 0.5 μM, 5 μM, and 50 μM.

As used herein, “cell” refers to the smallest structural unit of living matter capable of functioning autonomously, consisting of one or more nuclei, cytoplasm, and various organelles, all surrounded by a semipermeable membrane. Cells include all somatic cells obtained or derived from a living or deceased animal body at any stage of development as well as germ cells, including sperm and eggs (animal reproductive body consisting of an ovum or embryo together with nutritive and protective envelopes). Included are both general categories of cells: prokaryotes and eukaryotes. The cells contemplated for use in this invention include all types of cells from all organisms in all kingdoms: plans, animals, protists, fungi, archaebacteria and eubacteria. Stem cells are cells capable, by successive divisions, of producing specialized cells on many different levels. For example, hematopoietic stem cells produce both red blood cells and white blood cells. From conception until death, humans contain stem cells, but in adults their power to differentiate is reduced.

As used herein, the term “derived,” when referring to a biological sample, indicates the sample being obtained from the stated source at some point in time. For example, a biological sample derived from an organism can represent a primary biological sample obtained directly from the organism (i.e., unmodified), or can be modified, e.g., by introduction of a recombinant vector, by culturing under particular conditions, or immortalization.

As used herein, the phrase “at least one,” in reference to a list of one or more elements, should be understood to mean at least one element selected from any one or more of the elements in the list of elements, but not necessarily including at least one of each and every element listed within the list of elements and not excluding any combinations of elements in the list of elements. This definition also allows that elements may optionally be present other than the elements identified within the list of elements to which the phrase “at least one” refers, whether related or unrelated to those elements identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements).

As used herein, the term “biologically acceptable carrier” or “pharmaceutically acceptable carrier” refers to a pharmaceutically acceptable material, vehicle, or composition, such as a solid or liquid filler, binder, diluent, preservative, biocompatible solvent, disintegrating agent, lubricant, suspending agent, flavoring agent, encapsulating material, thickening agent, acid, surfactant, complexation agent, wetting agent, or any combination thereof. In some embodiments, each component is “pharmaceutically acceptable” in the sense of being compatible with the other ingredients of a pharmaceutical formulation, and suitable for use in contact with the organ or tissue of a subject without excessive toxicity, allergic response, irritation, immunogenicity, or other complications or problems. See, e.g., Remington: The Science and Practice of Pharmacy, 22nd ed.; Allen Ed.: Philadelphia, PA, 2012; Handbook of Pharmaceutical Excipients, 7th ed.; Rowe et al., Eds.; The Pharmaceutical Press and the American Pharmaceutical Association: 2012; Handbook of Pharmaceutical Additives, 3rd ed.; Ash and Ash Eds.; Gower Publishing Company: 2007; Pharmaceutical Preformulation and Formulation, 2nd ed.; Gibson Ed.; CRC Press LLC: Boca Raton, FL, 2009.

As used herein, the term “cosmetically acceptable carrier” refers to all carriers and/or excipients and/or diluents conventionally used in topical cosmetic compositions such as in particular in skin care preparations. Common examples include topical vehicles like creams and lotions, natural carrier oils, and advanced systems like liposomes that enhance ingredient penetration and stability. The right carrier is chosen based on the specific product, desired texture, and target skin conditions.

As used herein, the term “effective amount” refers to the amount of an active agent or a pharmaceutical composition that is sufficient to bring about an effect on treating, preventing, or ameliorating a disorder, disease, or condition of a subject in need thereof. The effective amount may vary by a person ordinarily skilled in the art, depending on excipient usage, routes of administration, the possibility of co-usage with other therapeutic treatment, or the condition to be treated, but the present disclosure is not limited thereto.

As used herein, the term “administer,” “administering” or “administration” refer to the placement of an active ingredient into a subject by a method or route which results in at least partial localization of the active ingredient at a desired site to produce the desired effect. For example, the active ingredient of the present disclosure may be administered to a subject by oral administration, injection, subcutaneous administration, intramuscular administration, topical administration, or nasal administration, but the present disclosure is not limited thereto.

As used herein, the term “treat,” “treating,” or “treatment” refers to obtaining a desired pharmacologic and/or physiologic effect. The effect may be prophylactic in terms of completely or partially preventing a disease or symptoms or conditions thereof or may be therapeutic in terms of completely or partially curing, alleviating, relieving, remedying, or ameliorating a disease or an adverse effect attributable to the disease or symptoms or conditions thereof.

As used herein, the term “prevent,” “preventing,” or “prevention” does not require that the disease state be completely thwarted. Rather, as used herein, the term “prevent,” “preventing,” or “prevention” refers to the ability of the skilled artisan to identify a population that is susceptible to disorders, such that administration of the extracellular vesicles of the present disclosure may occur prior to onset of a disease. The term does not imply that the disease state is completely avoided.

As used herein, the terms “patient” and “subject” are used interchangeably. The term “subject” refers to a mammal. The mammal includes, but not limited to, humans, non-human primates, canines, felines, murines, bovines, equines, porcines, sheeps, deers, wolves, foxes, and rabbits.

As used herein, the term “primed extracellular vesicles” or “bio-pulsed (bio-pulsing) extracellular vesicles” may be used interchangeably to refer to extracellular vesicles derived from cells cultured under a defined stimulation process. The cells may include stem cells such as mesenchymal stem cells (MSCs), and the defined stimulation process may include cultivation in a culture medium containing 2,3,5,4′-tetrahydroxystilbene-2-O-β-D-glucoside (THSG) and at least one selected from the group consisting of baicalin and bone morphogenetic protein 6 (hereinafter referred to as TRV bio-pulsing), but the present disclosure is not limited thereto.

As used herein, the term “fold change” is defined as primed extracellular vesicles divided by naïve extracellular vesicles on normalized counts.

In one aspect, the present disclosure provides a composition comprising primed extracellular vesicles and a biologically or pharmaceutically acceptable carrier, wherein the primed extracellular vesicles comprise an upregulated miRNA, and a fold change of the upregulated miRNA of the primed extracellular vesicles compared to miRNA of naturally occurring extracellular vesicles is greater than 1.

In some embodiments, the primed extracellular vesicles of the present disclosure are cultivated from mesenchymal stem cells.

In some embodiments, the mesenchymal stem cells of the present disclosure are selected from the group consisting of avian mesenchymal stem cells (hereinafter referred as, AMSCs), human umbilical cord mesenchymal stem cells (hereinafter referred as, hUC-MSCs), human placental mesenchymal stem cells (hereinafter referred as, hPLAMSCs), adipose-derived mesenchymal stem cells, bone-marrow mesenchymal stem cells, a dental pulp mesenchymal stem cells, gingival mesenchymal stem cells, and a combination thereof.

In some embodiments, the fold change of the upregulated miRNA of the primed extracellular vesicles of the present disclosure compared to the miRNA of the naturally occurring extracellular vesicles is greater than 1, the upregulated miRNA of the primed extracellular vesicles of the present disclosure is selected from the group consisting of miR-20a-5p, miR-26a-5p, miR-26b-5p, miR-29a-3p, miR-92a-3p, miR-200b-3p, miR-200c-3p, miR-204-5p, miR-210-3p, miR-214-3p, miR-218-5p, miR-222-3p, miR-335-5p, and a combination thereof.

In some embodiments, the fold change of the upregulated miRNA of the primed extracellular vesicles of the present disclosure compared to the miRNA of the naturally occurring extracellular vesicles is greater than 2, and the upregulated miRNA of the primed extracellular vesicles is selected from the group consisting of miR-29a-3p, miR-92a-3p, miR-200b-3p, miR-200c-3p, miR-204-5p, miR-214-3p, miR-335-5p, and a combination thereof.

In some embodiments, the fold change of the upregulated miRNA of the primed extracellular vesicles of the present disclosure compared to the miRNA of the naturally occurring extracellular vesicles is greater than 5, and the upregulated miRNA of the primed extracellular vesicles is selected from the group consisting of miR-200b-3p, miR-204-5p, miR-335-5p, and a combination thereof.

In some embodiments, the fold change of the upregulated miRNA of the primed extracellular vesicles of the present disclosure compared to the miRNA of the naturally occurring extracellular vesicles is greater than 13, and the upregulated miRNA of the primed extracellular vesicles is miR-335-5p.

In some embodiments, the fold change of a downregulated miRNA of the primed extracellular vesicles compared to miRNA of naturally occurring extracellular vesicles is less than 1.

In some embodiments, the downregulated miRNA of the primed extracellular vesicles of the present disclosure is selected from the group consisting of miR-30b-5p, miR-30e-5p, miR-31-5p, miR-34a-5p, miR-126-3p, miR-138-5p, miR-223-3p, and a combination thereof.

In some embodiments, the extracellular vesicle of the present disclosure is an apoptotic body, a microvesicle, an exosome, or any combination thereof. In some embodiments, the extracellular vesicle of the present disclosure is an exosome and/or a small extracellular vesicle. In some embodiments, the extracellular vesicles of the present disclosure comprise a polydisperse population of particles with a diameter ranging from about 30 nm to about 200 nm.

In some embodiments, the composition of the present disclosure is formulated into an injectable solution, hydrogel, scaffold composite, or implant coating.

In one aspect, the present disclosure further provides a method for preventing, treating, or ameliorating a bone defect or a dental defect comprising administering the composition of the present disclosure to a subject in need thereof.

In some embodiments, the dental defect is selected from the group consisting of long bone fracture, spinal fusion augmentation, osteoporotic fracture and non-union defect.

In some embodiments, the periodontal defect or the peri-implant defect is selected from the group consisting of alveolar ridge preservation, periodontal intrabony defect, furcation defect, and peri-implantitis-associated bone loss. In some embodiments, the composition of the present disclosure increases cementum attachment protein (hereinafter referred as CAP) expression while reducing dentin sialophosphoprotein (hereinafter referred as DSPP) expression in cementoblasts. In some embodiments, the composition of the present disclosure is applied as a surface coating on dental or orthopedic implants to enhance osseointegration and reduce peri-implant bone resorption.

In one embodiment, the administering of the present disclosure comprises topical administration.

In one embodiment, the administering of the present disclosure comprises local administration.

In one embodiment, the local administration comprises injection.

In some embodiments, the composition of the present disclosure exhibits enhanced osteogenic or cementogenic bioactivity.

In yet another aspect, the present disclosure further provides a method for producing the composition of the present disclosure and the method comprises cultivating a stem cell in a culture medium containing a Polygonum multiflorum Thunb extract and at least one selected from the group consisting of baicalin and bone morphogenetic protein 6 to obtain a cell culture; obtaining the extracellular vesicle from the cell culture; and mixing a biologically or pharmaceutically acceptable carrier with the extracellular vesicle.

In some embodiments, the Polygonum multiflorum Thunb extract comprises 2,3,4′,5-tetrahydroxystilbene-2-O-β-D-glucoside (hereinafter referred as THSG).

In some embodiments, the Polygonum multiflorum Thunb extract of the present disclosure has a concentration of about 0.1 μM to about 50 μM. In some embodiments, the baicalin of the present disclosure has a concentration of about 0.1 μM to about 50 μM. In some embodiments, the bone morphogenetic protein 6 of the present disclosure has a concentration of about 0.1 ng/ml to about 100 ng/mL.

In some embodiments, the cultivation of the MSCs of the present disclosure is performed for a time period ranging from 24 hours to 96 hours.

In some embodiments, the biologically or pharmaceutically acceptable carrier can be selected from the group consisting of inert diluents, dispersing and/or granulating agents, surface active agents and/or emulsifiers, disintegrating agents, binding agents, preservatives, buffering agents, lubricating agents, and/or oils.

In one aspect, the present disclosure further provides a use of the composition in manufacture of a medicament for preventing, treating, or ameliorating a bone defect or a dental defect.

In one aspect, the present disclosure further provides the composition for use in preventing, treating, or ameliorating a bone defect or a dental defect.

In some embodiments, the composition of the present disclosure may be formulated as a pharmaceutical composition including primed extracellular vesicles of the present disclosure and a pharmaceutically acceptable carrier.

In one aspect, the present disclosure further provides a non-therapeutic method for preventing, treating, or ameliorating a bone defect or a dental defect including administering the composition of the present disclosure to a subject in need thereof.

In some embodiments, the composition of the present disclosure may be formulated as a cosmetic composition or cosmeceutical composition including primed extracellular vesicles of the present disclosure and a cosmetically acceptable carrier.

Although the present disclosure is illustrated by specific embodiments and optional features, it is understood that modifications and variations of the concepts herein disclosed may be resorted to by those skilled in the art, and that such modifications and variations are considered to be within the scope of the present disclosure.

### EXAMPLE

Exemplary embodiments according to the present disclosure are further described in the following examples, which should not be construed to limit the scope of the present disclosure. The materials and methods used in the following examples are described in detail below. The materials used in the present disclosure but unannotated herein are commercially available.

### Example 1 Differential miRNA Profiling of BPEV-TBA by Next-Generation Sequencing

Extracellular vesicles (EVs) were isolated from mesenchymal stem cells (MSCs) cultured under standard conditions (naïve EVs) and from MSCs subjected to bio-pulsing with TBA (BPEV-TBA) (2,3,4′,5-tetrahydroxystilbene-2-O-β-D-glucoside (hereinafter referred as THSG±baicalin). Total RNA was extracted from purified EV preparations, and small RNA sequencing libraries were constructed. Next-generation sequencing (NGS) was performed to profile the expression of microRNAs (miRNAs). Raw sequencing data were normalized, and comparative analysis was conducted to determine fold-change differences between naïve EVs and BPEV-TBA.

As shown in Table 1, the NGS results revealed a distinct shift in the miRNA cargo of BPEV-TBA compared with naïve EVs. Multiple miRNAs associated with osteoblast differentiation and bone regeneration (e.g., hsa-miR-335-5p, hsa-miR-218-5p, hsa-miR-200c-3p, hsa-miR-210-3p, hsa-miR-26b-5p, and hsa-miR-214-3p) were significantly enriched following bio-pulsing.

TABLE 1
Differentially regulated miRNAs identified by NGS in BPEV-TBA
compared with Naïve MSC-EVs and the functional
relevance to bone regeneration. Table 1 shows an upregulated
hsa-miR profile (TRV/Naïve fold-change > 1).

| | Naïve | TRV | FC | | |
|---|---|---|---|---|---|
| miRNA | counts | counts | (TRV/Naïve) | Category | Functional note |
| hsa-miR-335-5p | 71 | 994 | 14 | OB+ | Enhances osteogenesis and |
| | | | | (osteoblast promoters) | bone regeneration |
| hsa-miR-218-5p | 0 | 1 | —* | OB+ | Promotes osteoblast |
| | | | | (osteoblast promoters) | differentiation |
| hsa-miR-200c-3p | 0 | 1 | —* | OB+ | Stimulates osteogenesis |
| | | | | (osteoblast promoters) | |
| hsa-miR-210-3p | 70 | 83 | 1.19 | OB+ | Hypoxia-induced |
| | | | | (osteoblast promoters) | osteogenesis |
| hsa-miR-26b-5p | 60 | 62 | 1.03 | OB+ | Promotes osteoblast |
| | | | | (osteoblast promoters) | differentiation |
| hsa-miR-214-3p | 22 | 53 | 2.41 | OC+ | Promotes osteoclast activity, |
| | | | | (osteoclast promoters) | inhibits osteoblasts |

*undefined due to zero denominator

As shown in Table 2, the NGS results revealed a distinct shift in the miRNA cargo of BPEV-TBA compared with naïve EVs. Several miRNAs known to inhibit osteogenesis (e.g., hsa-miR-30b-5p and hsa-miR-138-5p) were downregulated in BPEV-TBA relative to naïve EVs.

TABLE 2
Differentially regulated miRNAs identified by NGS in BPEV-TBA
compared with Naïve MSC-EVs and the functional
relevance to bone regeneration. Table 2 shows a downregulated
hsa-miR profile (TRV/Naïve fold-change < 1).

| | Naïve | TRV | FC | | |
|---|---|---|---|---|---|
| miRNA | counts | counts | (TRV/Naïve) | Category | Functional note |
| hsa-miR-30b-5p | 44 | 30 | 0.68 | OB− | Suppresses osteogenesis |
| | | | | (osteoblast inhibitors) | |
| hsa-miR-138-5p | 4 | 2 | 0.5 | OB− | Strong inhibitor of |
| | | | | (osteoblast inhibitors) | osteogenesis |
| hsa-miR-30e-5p | 77 | 43 | 0.56 | OB− | Inhibits osteogenesis |
| | | | | (osteoblast inhibitors) | |
| hsa-miR-34a-5p | 850 | 752 | 0.88 | OC− | Strong anti-resorptive |
| | | | | (osteoclast inhibitors) | miRNA; increases bone |
| | | | | | mass |
| hsa-miR-223-3p | 7 | 0 | 0 | OC− | Dual role |
| | | | | (osteoclast inhibitors) | (context-dependent, often |
| | | | | | pro-osteoclast) |
| hsa-miR-31-5p | 1846 | 924 | 0.5 | OC+ | Regulates cytoskeleton for |
| | | | | (osteoclast promoters) | bone resorption |

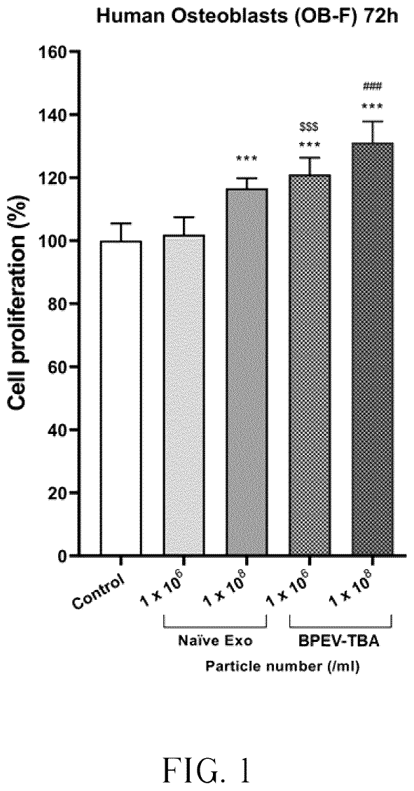

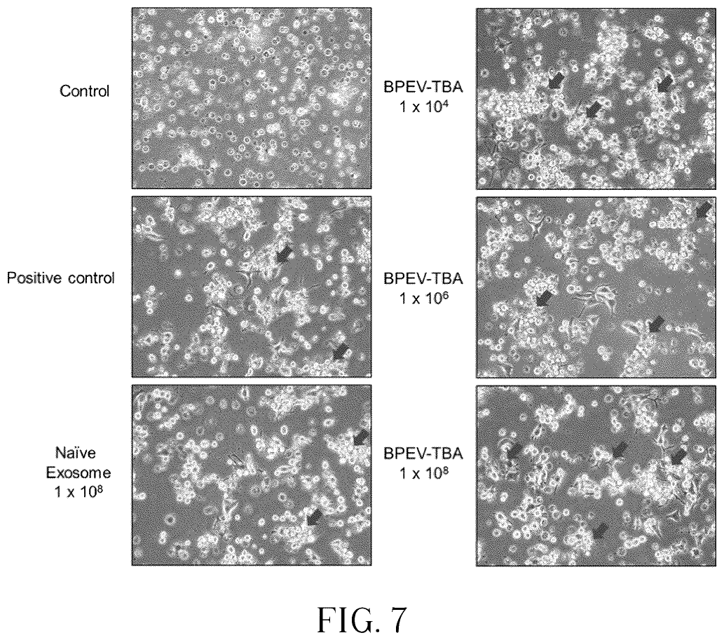

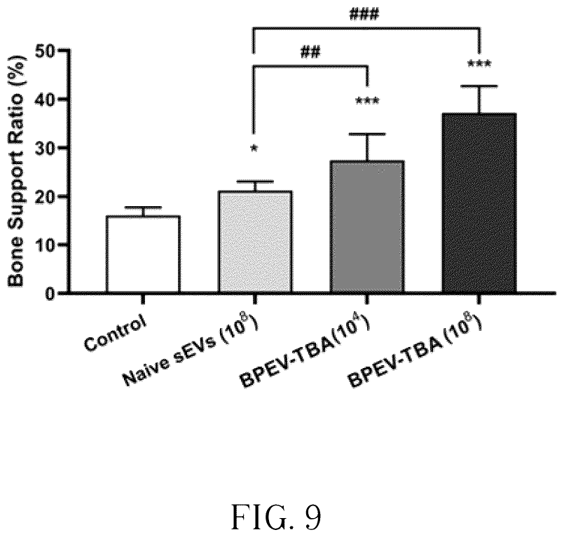

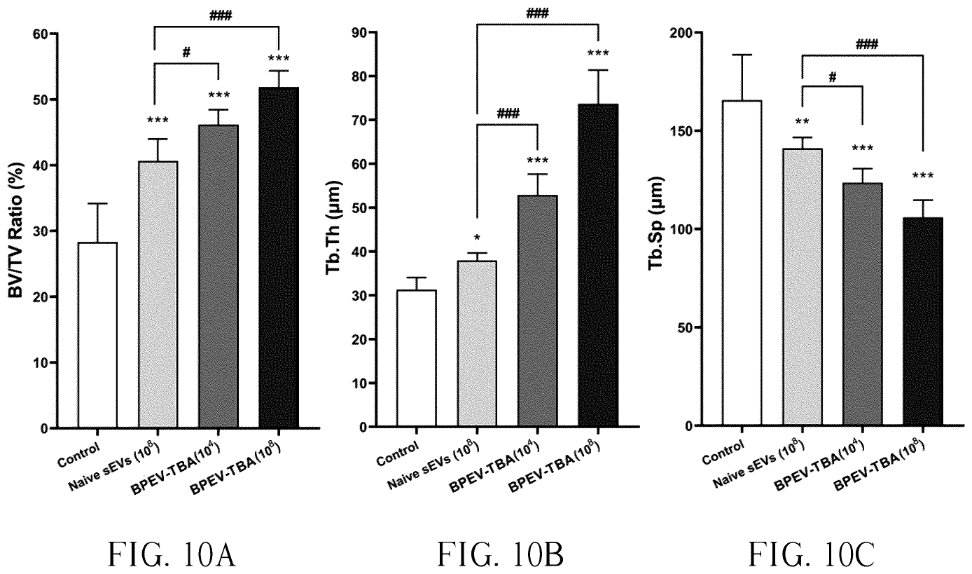

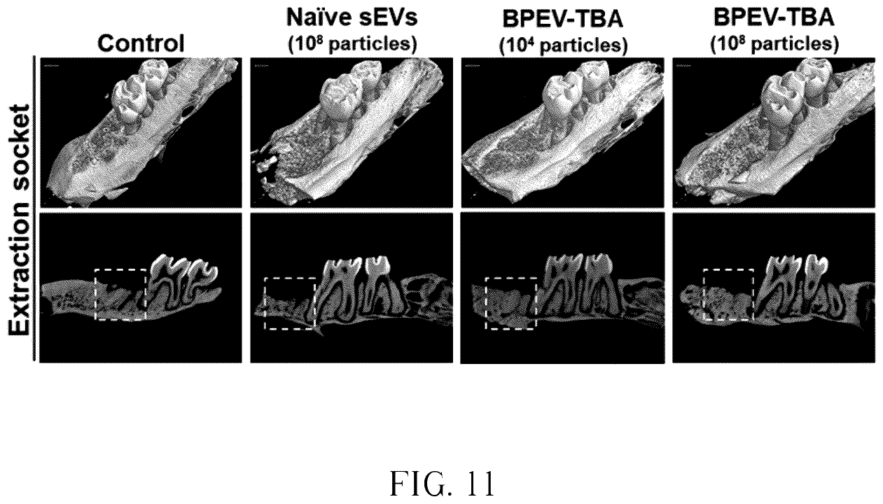

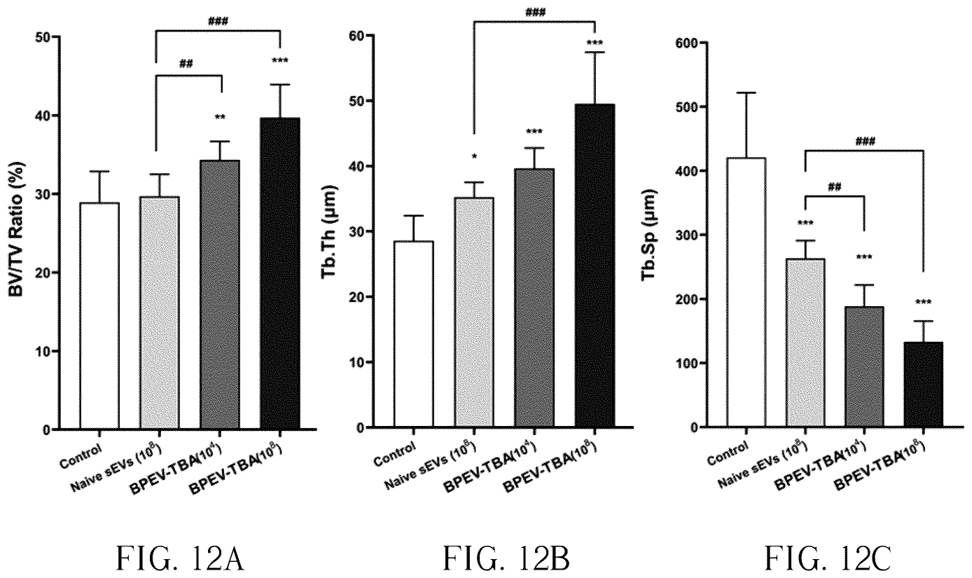

Taken together, the NGS data demonstrates that TBA bio-pulsing induces a selective enrichment of pro-osteogenic and anti-resorptive miRNAs while downregulating inhibitors of bone formation. This molecular profile provides a mechanistic basis for the observed osteogenic effects of BPEV-TBA in promoting osteoblast proliferation, differentiation, and mineralization, as shown in FIG. 1 to FIG. 12C.

### Example 2. Differential miRNA Profiling of BPEV-TBMP6 by Next-Generation Sequencing

Extracellular vesicles (EVs) were harvested from mesenchymal stem cells (MSCs) cultured under standard conditions (naïve EVs) and from MSCs subjected to bio-pulsing with TBMP6 (BPEV-TBMP6) (2,3,4′,5-tetrahydroxystilbene-2-O-β-D-glucoside (hereinafter referred as THSG)±bone morphogenetic protein-6 (hereinafter referred as BMP-6)).

Total RNA was extracted, and small RNA sequencing libraries were prepared. Next-generation sequencing (NGS) was performed to quantify miRNA expression profiles. Normalized read counts were compared between naïve EVs and BPEV-TBMP6, and fold-change differences were calculated.

As summarized in Table 3, bio-pulsing with TBMP6 induced a distinct shift in the EV miRNA cargo. Several miRNAs categorized as anti-cementogenic or anti-mineralization regulators (including hsa-miR-204-5p) exhibited altered expression patterns, with upregulation activity relative to naïve EVs.

TABLE 3
Differentially regulated miRNAs identified by NGS in
BPEV-TBMP6 compared with naive MSC-EVs and their functional
relevance to cementogenesis and periodontal regeneration.
Table 3 shows an upregulated hsa-miR profile
(TRV/Naïve fold-change > 1).

| | Naïve | TRV | FC | | |
|---|---|---|---|---|---|
| miRNA | counts | counts | (TRV/Naïve) | Category | Functional note |
| hsa-miR-222-3p | 8 | 15 | 1.88 | Angiogenesis inhibitors | Anti-osteogenic, |
| | | | | | anti-angiogenic. |
| hsa-miR-204-5p | 3 | 20 | 6.67 | Anti-cementogenesis/ | Blocks osteogenesis, |
| | | | | mineralization | angiogenesis inhibitor |
| hsa-miR-29a-3p | 60 | 129 | 2.15 | ECM remodeling/PDL | ECM mineralization, |
| | | | | | collagen regulation. |
| hsa-miR-92a-3p | 24 | 52 | 2.17 | ECM remodeling/PDL | ECM mineralization, |
| | | | | | collagen regulation. |
| hsa-miR-20a-5p | 21 | 34 | 1.62 | ECM remodeling/PDL | Anti-osteoclast, inhibits |
| | | | | | NF-κB |
| hsa-miR-200b-3p | 2 | 15 | 7.5 | Pro-cementogenesis/ | ECM mineralization, |
| | | | | mineralization | collagen regulation |
| hsa-miR-200c-3p | 32 | 78 | 2.44 | Pro-cementogenesis/ | ECM mineralization, |
| | | | | mineralization | collagen regulation |
| hsa-miR-26b-5p | 12 | 23 | 1.92 | Pro-cementogenesis/ | Wnt/BMP activator, |
| | | | | mineralization | pro-osteogenic |
| hsa-miR-26a-5p | 490 | 877 | 1.79 | Pro-cementogenesis/ | Wnt/BMP activator, |
| | | | | mineralization | pro-osteogenic |
| hsa-miR-218-5p | 3 | 5 | 1.67 | Pro-cementogenesis/ | Wnt/BMP activator, |
| | | | | mineralization | pro-osteogenic |

As summarized in Table 4, BPEV-TBMP6 displayed an enrichment of miRNAs associated with extracellular matrix (ECM) remodeling and periodontal ligament (PDL) regeneration, such as hsa-miR-29a-3p, hsa-miR-92a-3p, and hsa-miR-20a-5p.

TABLE 4
Differentially regulated miRNAs identified by NGS in
BPEV-TBMP6 compared with naive MSC-EVs and their functional
relevance to cementogenesis and periodontal regeneration.
Table 4 shows a downregulated hsa-miR profile
(TRV/Naïve fold-change < 1).

| | Naïve | TRV | FC | | |
|---|---|---|---|---|---|
| miRNA | counts | counts | (TRV/Naïve) | Category | Functional note |
| hsa-miR-126-3p | 59 | 39 | 0.67 | Angiogenesis | Endothelial pro-angiogenic, |
| | | | | promoters | VEGF activator. |

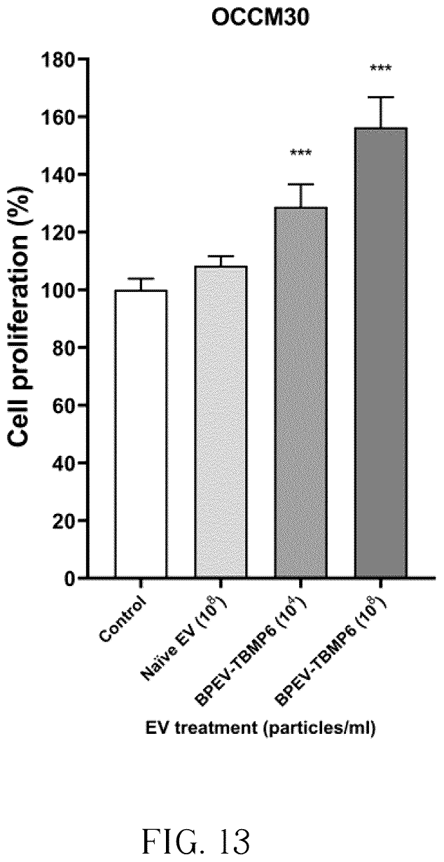

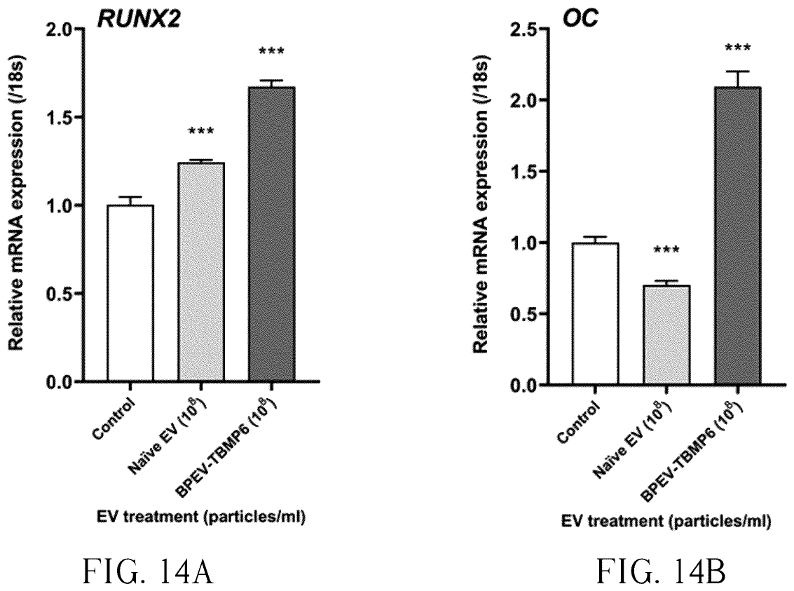

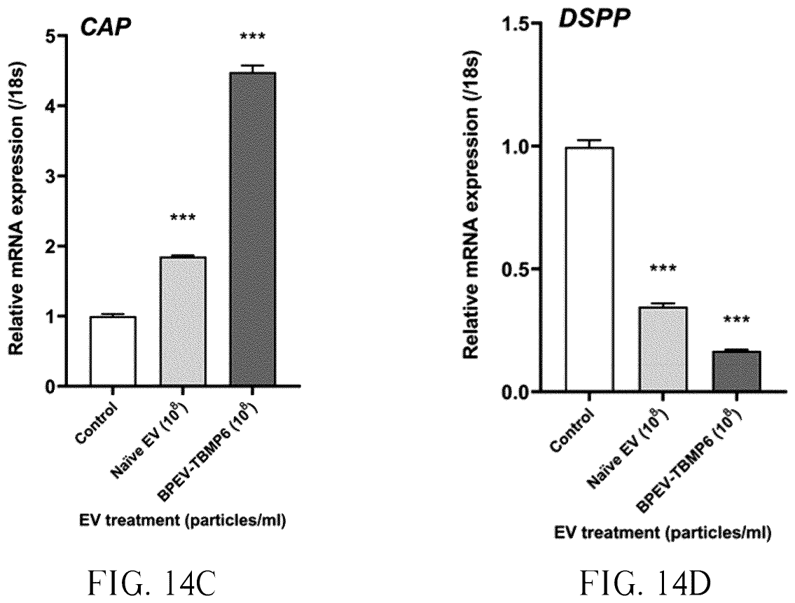

Taken together, these results demonstrate that TBMP6 bio-pulsing reprograms MSC-EV cargo toward a profile that supports cementoblast activity, ECM mineralization, and periodontal tissue regeneration. The molecular data provides a mechanistic basis for the observed cementogenic effects of BPEV-TBMP6 on cementoblast proliferation and mineralized matrix formation, as shown in FIG. 13 to FIG. 14D.

### Example 3 In Vitro Functional Effects of BPEV-TBA on Bone Remodeling

Bio-pulsed extracellular vesicles derived from TBA-stimulated mesenchymal stem cells (BPEV-TBA) were evaluated for their capacity to regulate bone remodeling by acting on both osteoblasts and osteoclasts. These experiments collectively demonstrate that BPEV-TBA promotes bone formation and suppresses bone resorption, supporting its application in orthopedic and dental regenerative therapies.

### 3.1 Osteoblast Proliferation and Migration

Primary human osteoblasts (OB-F) and human alveolar bone-derived cells were cultured in standard medium supplemented with either control medium, naïve MSC-derived EVs, or bio-pulsed EVs (BPEV-TBA) at concentrations ranging from 1×10⁶ to 1×10⁸ particles/mL. Proliferative and migratory responses were assessed to determine the pro-regenerative potential of BPEV-TBA.

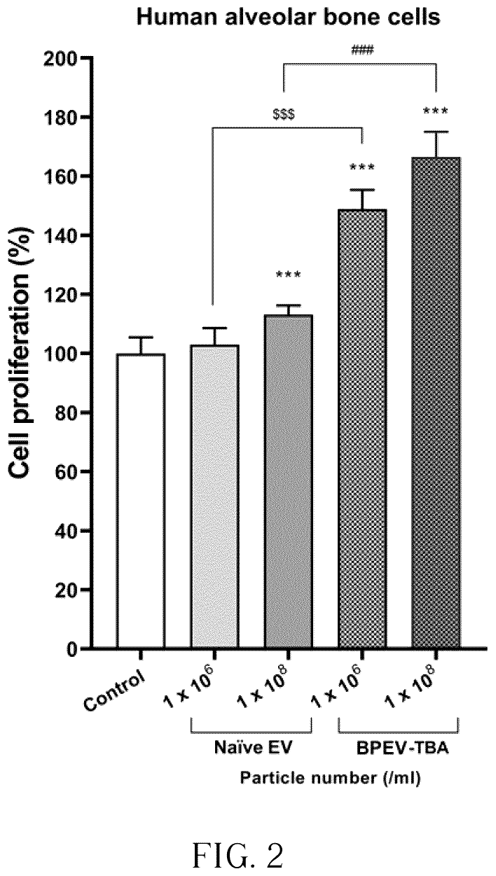

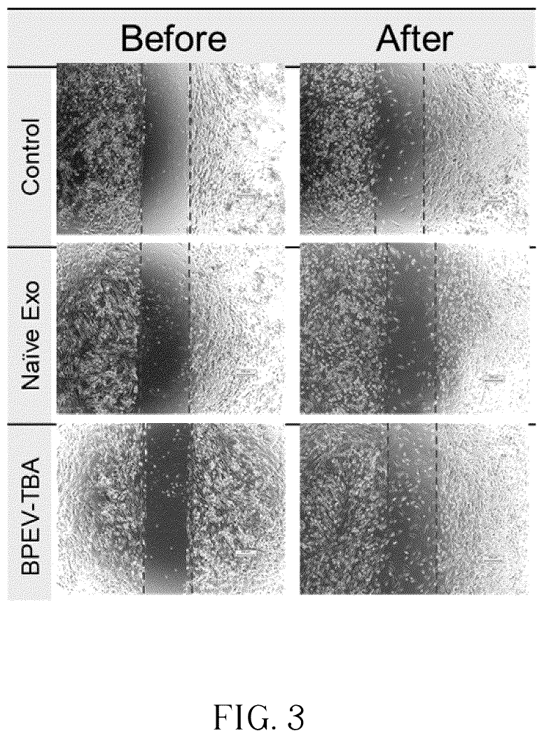

Cell proliferation assays: After 72 hours of treatment, cell proliferation was quantified using the CCK-8 assay. As shown in FIG. 1 (OB-F cells) and FIG. 2 (alveolar bone cells), BPEV-TBA significantly enhanced proliferation in a dose-dependent manner compared to both naïve EVs and untreated controls. At the highest dose, BPEV-TBA increased osteoblast proliferation by up to 1.5-2 fold relative to baseline.

Scratch wound assay: To evaluate migratory ability, osteoblast monolayers were seeded in ibidi Culture-Inserts to generate standardized gaps. After 24 hours of incubation, FIG. 3 demonstrates accelerated wound closure in BPEV-TBA-treated groups, while gaps persisted in control and naïve EV groups.

These findings indicate that BPEV-TBA promotes both the expansion and migration of osteoblasts, two essential processes underlying bone regeneration. Such dual activity supports the application of BPEV-TBA in clinical contexts such as alveolar ridge preservation, periodontal regeneration, and fracture repair, where rapid recruitment of functional osteoblasts is critical for effective healing.

### 3.2 Osteoblast Differentiation and Mineralization

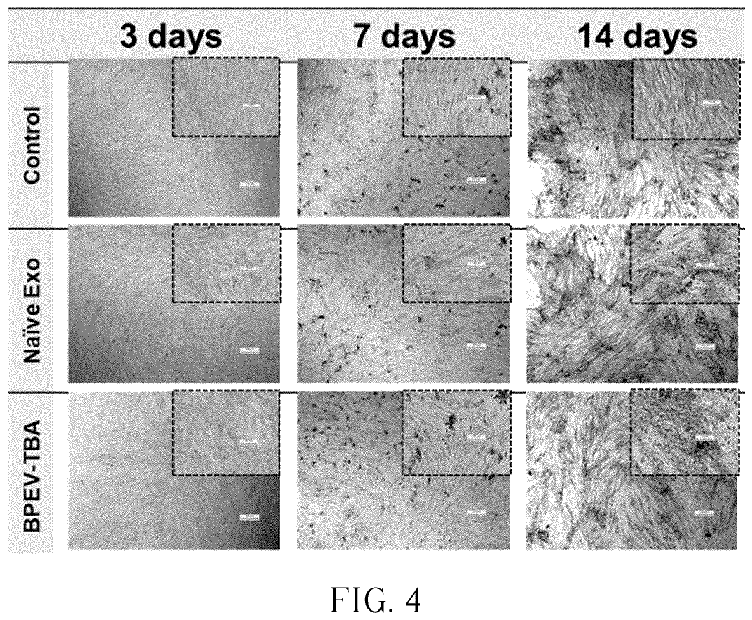

ALP activity: Alkaline phosphatase (ALP) staining, an early marker of osteogenic differentiation, demonstrated enhanced activity in BPEV-TBA-treated groups. As shown in FIG. 4, osteoblasts cultured with BPEV-TBA exhibited markedly stronger ALP staining at 3, 7, and 14 days, compared to naïve EVs and untreated controls.

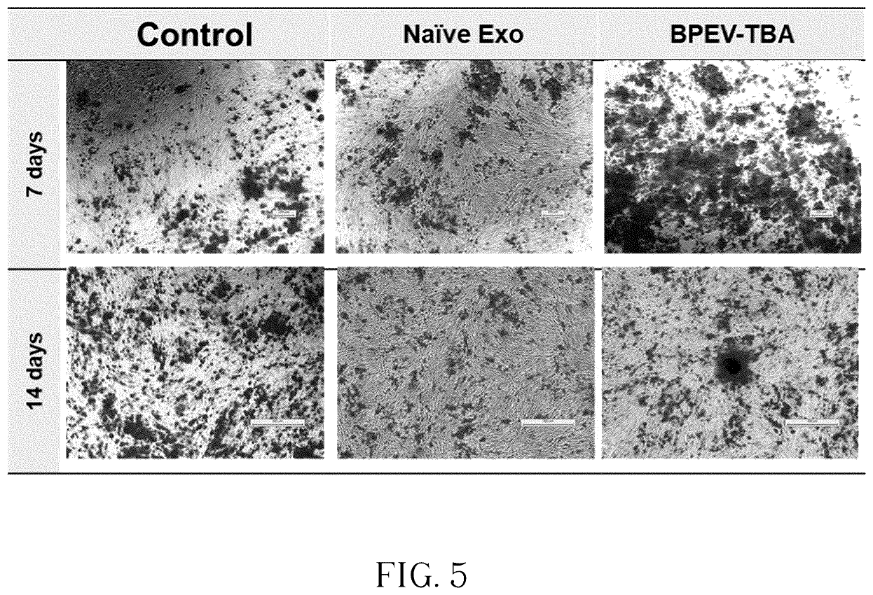

Matrix mineralization: Alizarin Red staining confirmed that BPEV-TBA promoted extensive calcium nodule formation. Representative images in FIG. 5 reveal that mineralized matrix deposition was substantially greater in the BPEV-TBA group at both 7 and 14 days relative to naïve EVs and controls.

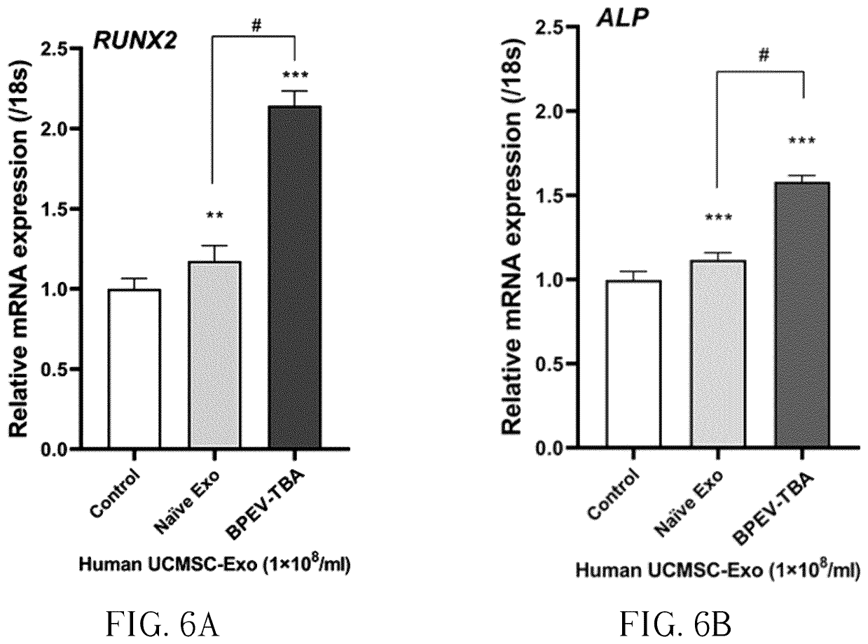

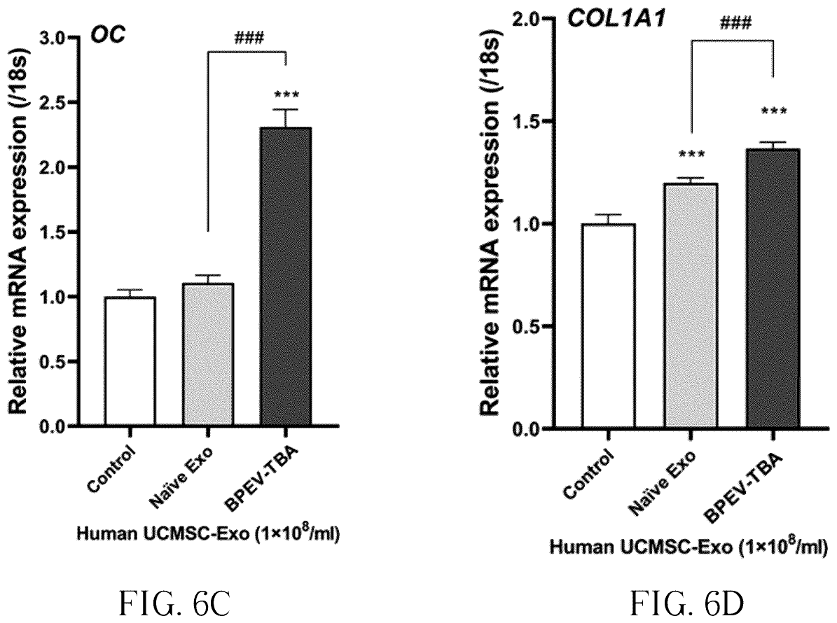

Gene expression: Quantitative PCR analysis after 3 days of osteogenic induction (as shown in FIG. 6A, FIG. 6B, FIG. 6C and FIG. 6D) showed that BPEV-TBA significantly upregulated the transcription of osteogenic marker genes, including RUNX2, ALP, osteocalcin (OC), and COL1A1, compared to naïve EVs and untreated controls.

Collectively, these results demonstrate that BPEV-TBA accelerates osteoblast differentiation, enhances matrix mineralization, and activates osteogenic gene expression programs. This indicates that BPEV-TBA promotes robust and functional bone matrix deposition, thereby supporting its application in bone regeneration.

### 3.3 Osteoclast Differentiation Inhibition

The human monocyte cell line U937 was induced to differentiate into osteoclast-like cells using PMA combined with vitamin D3. Cells were exposed to control medium, naïve EVs, or BPEV-TBA (10⁴-10⁸ particles/mL).

Morphological observation: In positive control groups, multinucleated osteoclast-like cells formed after 7 days. Naïve EVs modestly reduced osteoclast formation, while BPEV-TBA suppressed osteoclastogenesis in a dose-dependent fashion. At the highest dose (10⁸ particles/mL), very few multinucleated cells were observed (as shown in FIG. 6A, FIG. 6B, FIG. 6C and FIG. 6D).

These data demonstrate that BPEV-TBA not only enhances osteoblast activity but also reduces osteoclastogenesis, thereby shifting the balance of bone remodeling toward net bone formation.

### 3.4 Mechanistic Link: MiRNA Profile and Functional Outcomes

The NGS-based miRNA profiling (see Example 2, Table 3 and Table 4) revealed that BPEV-TBA is enriched in pro-osteogenic miRNAs (e.g., miR-335-5p, miR-218-5p, miR-200c) while suppressing inhibitors of osteogenesis (e.g., miR-30 family, miR-221). In addition, BPEV-TBA downregulated miRNAs linked to osteoclast differentiation (e.g., miR-21, miR-214). This molecular signature aligns with the observed functional results in osteoblast and osteoclast assays, indicating that BPEV-TBA reprograms EV cargo toward a bone-regenerative phenotype.

Collectively, the results of this Example demonstrate that BPEV-TBA:
1. Promotes osteoblast proliferation, migration, differentiation, and mineralized matrix formation.
2. Suppresses osteoclast differentiation and activity, reducing bone resorption potential.
3. Exhibits a miRNA profile consistent with pro-osteogenic and anti-resorptive functions.

These findings provide strong mechanistic and functional evidence that BPEV-TBA serves as a dual-action, cell-free biologic capable of restoring bone homeostasis, supporting its application in orthopedic bone repair, alveolar ridge preservation, periodontal regeneration, and implantology.

### Example 4 In Vivo Evaluation of BPEV-TBA in Periodontal and Extraction Socket Bone Regeneration

Bio-pulsed extracellular vesicles derived from TBA-stimulated mesenchymal stem cells (BPEV-TBA) were evaluated for their regenerative potential in vivo using a rat model of periodontal defects and tooth extraction sockets.

### Experimental Design

1. Male Sprague-Dawley rats (6-8 weeks old, 180-230 g) were randomly assigned to four groups: (1) untreated control, (2) naïve MSC-derived EVs (1×10⁸ particles), (3) BPEV-TBA at a low dose (1×10⁴ particles), and (4) BPEV-TBA at a high dose (1×10⁸ particles).
2. Standardized periodontal defects (2.5×2.0× 1.0 mm) were surgically created adjacent to the maxillary second molars (M2). In parallel, extraction sockets were prepared by atraumatic removal of the maxillary first molars (M1). Treatments were administered immediately following defect creation by incorporating EVs into a Matrigel carrier, followed by primary closure of the flaps.

### Micro-CT Analysis

At 4 weeks post-surgery, the maxillae were harvested and subjected to high-resolution microcomputed tomography (μCT, voxel size 18 μm). Parameters including bone volume fraction (BV/TV), trabecular thickness (Tb.Th), and bone mineral density (BMD) were quantified.

### Results

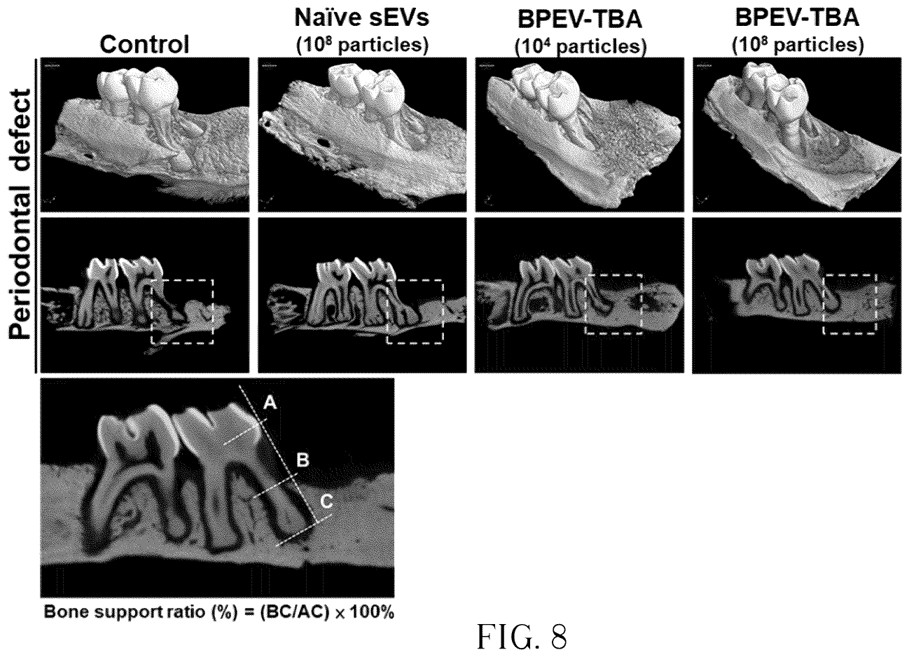

1. As summarized in FIG. 8 to FIG. 12C (micro-CT analysis), both periodontal defects and extraction sockets treated with BPEV-TBA exhibited enhanced bone regeneration compared with naïve EVs and untreated controls.
2. In periodontal defects, the bone support ratio was significantly higher in the BPEV-TBA high-dose group relative to naïve EV treatment.
3. In extraction sockets, BPEV-TBA treatment promoted greater alveolar bone fill and improved trabecular architecture in a dose-dependent manner.
4. These data demonstrate that BPEV-TBA not only increases bone volume and density but also improves microarchitectural parameters associated with functional bone regeneration.

This in vivo study provides evidence that bio-pulsing with TBA reprograms MSC-derived EVs to enhance periodontal and alveolar bone healing, offering a novel, cell-free therapeutic approach for oral and maxillofacial bone regeneration.

### Example 5 Effects of BPEV-TBMP6 on Cementoblast Proliferation and Cementogenic Gene Expression

To evaluate the potential of bio-pulsed extracellular vesicles stimulated with bone morphogenetic protein 6 (BPEV-TBMP6) in cementum regeneration, murine cementoblast OCCM30 cells were used as an in vitro model system. OCCM30 cells represent a well-characterized cementoblast cell line widely applied for the study of cementogenesis and periodontal tissue repair.

### Experimental Setup

1) OCCM30 cells were seeded at appropriate densities and cultured in standard growth medium. After attachment, cells were exposed to either:
   1. Control medium (vehicle only),
   2. Naïve MSC-derived EVs (1×10⁸ particles/mL), or
   3. BPEV-TBMP6 at doses of 1×10⁶ or 1×10⁸ particles/mL.
2) Cells were maintained for either 48 hours (for proliferation assays) or 3 days under mineralization-inducing conditions (for gene expression analysis).
3) Cell proliferation was assessed by CCK-8 assay.
4) Gene expression of cementogenic markers was quantified by qPCR, normalized to 18s rRNA. Target genes included:
   1. RUNX2 (osteogenic transcription factor),
   2. Osteocalcin (OC) (late osteogenic marker),
   3. Cementum attachment protein (CAP) (cementoblast-specific matrix protein),
   4. Dentin sialophosphoprotein (DSPP) (dentin/cementum-associated matrix protein).

### Results

### Cell Proliferation (as Shown in FIG. 13):

After 48 hours of treatment, BPEV-TBMP6 significantly enhanced OCCM30 proliferation compared to both control and naïve EV groups. A dose-dependent effect was observed, with the higher dose (1×10⁸ particles/mL) producing up to a 1.6-fold increase in cell proliferation relative to untreated controls. This demonstrates that BPEV-TBMP6 provides superior proliferative support to cementoblasts, a critical prerequisite for cementum repair.

### Cementogenic Gene Expression (as Shown in FIG. 14A to FIG. 14D):

After 3 days of mineralization induction, BPEV-TBMP6 markedly modulated cementoblast gene expression profiles:
1. RUNX2 expression was significantly elevated, indicating enhanced transcriptional commitment to osteo/cementogenic lineage differentiation.
2. Osteocalcin (OC) expression increased robustly, reflecting promotion of late-stage mineralizing activity.
3. CAP expression showed strong upregulation (up to four-fold relative to controls), confirming a cementoblast-specific response and functional cementogenesis bias.
4. DSPP expression, in contrast, was markedly suppressed compared to naïve EVs and controls, suggesting a selective reprogramming of EV cargo that prioritizes cementum formation over dentin-like matrix deposition.

### Interpretation and Relevance

These results demonstrate that BPEV-TBMP6 not only stimulates cementoblast proliferation but also reprograms gene expression toward a cementogenesis-favorable profile, characterized by enhanced RUNX2, OC, and CAP while suppressing DSPP. Such selective gene regulation supports targeted cementum regeneration, distinguishing BPEV-TBMP6 from naïve EVs and highlighting its engineered functionality.

This dual effect—accelerated expansion of cementoblasts and activation of cementum-specific gene expression—is highly advantageous in periodontal regenerative applications, including:
1. Guided tissue regeneration (GTR),
2. Periodontal defect repair,
3. Cementum-dentin complex restoration,
4. Dental implant integration through enhanced cementum formation.

Accordingly, BPEV-TBMP6 represents a novel and potent therapeutic candidate for dental and periodontal regenerative medicine, with translational potential for incorporation into scaffolds, hydrogels, or injectable delivery systems.

The present disclosure has been described with embodiments thereof, and it is understood that various modifications, without departing from the scope of the present disclosure, are in accordance with the embodiments of the present disclosure. Hence, the embodiments described are intended to cover the modifications within the scope of the present disclosure, rather than to limit the present disclosure. The scope of the claims therefore should be accorded the broadest interpretation so as to encompass all such modifications.

What is claimed is:

1. A composition comprising primed extracellular vesicles and a biologically or pharmaceutically acceptable carrier, wherein the primed extracellular vesicles comprise an upregulated miRNA, and a fold change of the upregulated miRNA of the primed extracellular vesicles compared to miRNA of naturally occurring extracellular vesicles is greater than 1.

2. The composition of claim 1, wherein the primed extracellular vesicles are cultivated from mesenchymal stem cells.

3. The composition of claim 2, wherein the mesenchymal stem cells are selected from the group consisting of avian mesenchymal stem cells, human umbilical cord mesenchymal stem cells, human placental mesenchymal stem cells, adipose-derived mesenchymal stem cells, bone-marrow mesenchymal stem cells, dental pulp mesenchymal stem cells, gingival mesenchymal stem cells, and a combination thereof.

4. The composition of claim 1, wherein the upregulated miRNA of the primed extracellular vesicles is selected from the group consisting of miR-20a-5p, miR-26a-5p, miR-26b-5p, miR-29a-3p, miR-92a-3p, miR-200b-3p, miR-200c-3p, miR-204-5p, miR-210-3p, miR-214-3p, miR-218-5p, miR-222-3p, miR-335-5p, and a combination thereof.

5. The composition of claim 4, wherein the fold change of the upregulated miRNA of the primed extracellular vesicles compared to the miRNA of the naturally occurring extracellular vesicles is greater than 2, and the upregulated miRNA of the primed extracellular vesicles is selected from the group consisting of miR-29a-3p, miR-92a-3p, miR-200b-3p, miR-200c-3p, miR-204-5p, miR-214-3p, miR-335-5p, and a combination thereof.

6. The composition of claim 5, wherein the fold change of the upregulated miRNA of the primed extracellular vesicles compared to the miRNA of the naturally occurring extracellular vesicles is greater than 5, and the upregulated miRNA of the primed extracellular vesicles is selected from the group consisting of miR-200b-3p, miR-204-5p, miR-335-5p, and a combination thereof.

7. The composition of claim 6, wherein the fold change of the upregulated miRNA of the primed extracellular vesicles compared to the miRNA of the naturally occurring extracellular vesicles is greater than 13, and the upregulated miRNA of the primed extracellular vesicles is miR-335-5p.

8. The composition of claim 1, wherein a fold change of a downregulated miRNA of the primed extracellular vesicles compared to miRNA of naturally occurring extracellular vesicles is less than 1.

9. The composition of claim 5, wherein the downregulated miRNA of the primed extracellular vesicles is selected from the group consisting of miR-30b-5p, miR-30e-5p, miR-31-5p, miR-34a-5p, miR-126-3p, miR-138-5p, miR-223-3p, and a combination thereof.

10. The composition of claim 1, wherein the extracellular vesicle is an apoptotic body, a microvesicle, an exosome, or any combination thereof.

11. The composition of claim 1, wherein the extracellular vesicles comprise a polydisperse population of particles with a diameter ranging from about 30 nm to about 200 nm.

12. The composition of claim 1, wherein the composition is formulated into an injectable solution, hydrogel, scaffold composite, or implant coating.

13. A method for preventing, treating, or ameliorating a bone defect or a dental defect comprising administering the composition of claim 1 to a subject in need thereof.

14. The method of claim 13, wherein the bone defect is selected from the group consisting of long bone fracture, spinal fusion augmentation, osteoporotic fracture and non-union defect.

15. The method of claim 13, wherein the dental defect is selected from the group consisting of alveolar ridge preservation, periodontal intrabony defect, furcation defect, and peri-implantitis-associated bone loss.

16. The method of claim 13, wherein the administering comprises topical administration.

17. The method of claim 13, wherein the administering comprises local administration.

18. The method of claim 13, wherein the composition exhibits enhanced osteogenic or cementogenic bioactivity.

19. A method for producing the composition of claim 1, comprising:
cultivating a stem cell in a culture medium containing a Polygonum multiflorum Thunb extract and at least one selected from the group consisting of baicalin and bone morphogenetic protein 6 to obtain a cell culture;
obtaining the extracellular vesicle from the cell culture; and
mixing a biologically or pharmaceutically acceptable carrier with the extracellular vesicle.

20. The method of claim 19, wherein the stem cell is mesenchymal stem cell.

21. The method of claim 20, wherein the mesenchymal stem cell is selected from the group consisting of avian mesenchymal stem cells, human umbilical cord mesenchymal stem cells, human placental mesenchymal stem cells, adipose-derived mesenchymal stem cells, bone-marrow mesenchymal stem cells, dental pulp mesenchymal stem cells, gingival mesenchymal stem cells, and a combination thereof.

22. The method of claim 19, wherein the Polygonum multiflorum Thunb extract comprises 2,3,4′,5-tetrahydroxystilbene-2-O-β-D-glucoside (THSG).

23. The method of claim 19, wherein the Polygonum multiflorum Thunb extract has a concentration of about 0.1 μM to about 50 μM.

24. The method of claim 19, wherein the cultivation of the stem cell is performed for a time period ranging from 24 hours to 96 hours.

25. The method of claim 19, wherein the composition is formulated into an injectable solution, hydrogel, scaffold composite, or implant coating.
